(19) United States
(12) Patent Application Publication
(10) Pub. No.: US 2015/0073442 A1
(43) Pub. Date: Mar. 12, 2015

(54) SUTURE PASSER AND METHOD FOR HIP LABRUM REPAIR

(71) Applicants: Justin D. SALIMAN, Los Angeles, CA (US); John G. MCCUTCHEON, Menlo Park, CA (US)

(72) Inventors: Justin D. SALIMAN, Los Angeles, CA (US); John G. MCCUTCHEON, Menlo Park, CA (US)

(21) Appl. No.: 14/546,942

(22) Filed: Nov. 18, 2014

Related U.S. Application Data
(63) Continuation-in-part of application No. 13/759,006, filed on Feb. 4, 2013.
(60) Provisional application No. 61/905,762, filed on Nov. 18, 2013.

(51) Int. Cl. A61B 17/04

(52) U.S. Cl. CPC A61B 17/0469
USPC 606/144

(57) ABSTRACT

Suture passer devices and methods for repair tissue using them. In particular, described herein are suture passer devices having a tissue penetrating distal end that can be used to repair tissue such as the hip labrum. In general, such devices may be used to manipulate, including move or align tissues, and to suture them.

### CROSS REFERENCE TO RELATED APPLICATIONS

This patent application claims priority as a continuation-in-part of U.S. patent application Ser. No. 13/759,006, titled “SUTURE PASSERS,” filed on Feb. 4, 2013, Publication No. US-2014-0222034-A1. This patent application also claims priority to U.S. Provisional Patent Application No. 61/905,762, filed on Nov. 18, 2013, titled “SUTURE PASSER AND METHOD FOR HIP LABRUM REPAIR”. Each of these patent applications is herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Suturing of tissue during surgical procedures is time consuming and can be particularly challenging in difficult to access body regions and regions that have limited clearance, such as regions partially surrounded or partially covered by bone and/or immediately adjacent to bone. For many surgical procedures, it is necessary to make a large opening in the human body to expose the area requiring surgical repair. However, in many cases, accessing the tissue in this manner is undesirable, increasing recovery time, and exposing the patient to greater risk of infection.

Suturing instruments (“suture passers” or “suturing devices”) have been developed to assist in accessing and treating internal body regions, and to generally assist a physician in repairing tissue. Although many such devices are available for endoscopic and/or percutaneous use, these devices suffer from a variety of problems, including limited ability to navigate and be operated within the tight confines of the body, risk of injury to adjacent structures, problems controlling the position and/or condition of the tissue before, during, and after passing the suture, as well as problems with the reliable functioning of the suture passer.

For example, some surgical instruments used in endoscopic procedures are limited by the manner in which they access the areas of the human body in need of repair. In particular, the instruments may not be able to access tissue or organs located deep within the body or that are in some way obstructed. In addition, many of the instruments are limited by the way they grasp tissue, apply a suture, or recapture the needle and suture. Furthermore, many of the instruments are complicated and expensive to use due to the numerous parts and/or subassemblies required to make them function properly. Suturing remains a delicate and time-consuming aspect of most surgeries, including those performed endoscopically.

A non-exhaustive list of difficult to access, and therefore difficult to properly repair, tissues includes the hip labrum, meniscus of the knee, the tendons and ligaments of the shoulder (e.g., rotator cuff), and non-bony spinal tissues (including the disc annulus). Any such structures may benefit from the devices and methods described herein. For example, a healthy hip is shown in FIG. 1A. In the healthy hip, the labrum 202 (i.e., the piece of connective tissue around the rim of the acetabulum or hip socket) is attached directly to the acetabulum 204. The head of the femur 206 sits within the labrum against the acetabulum 204 and moves relative to the acetabulum 204. The labrum 202 provides a seal for the hip joint, thus maintaining fluid pressure inside the joint and providing the joint cartilage with nutrition. However, referring to FIG. 1B, if the labrum 202 is torn (e.g., such as with chrondo-labral separation 210), the seal between the femur 206 and the acetabulum 204 can be disrupted, causing a shift in the hip center of rotation, pain, wear and tear of the hip joint, and an increased risk for early degenerative arthritis. Accordingly, there is a need for methods, devices and systems for suturing tissue in difficult to access regions of the body including the joints, such as the hip labrum.

Furthermore, when such difficult-to-access areas of the body, such as the hip labrum, are sutured, it is often necessary to cut other tissue, such as preforming capsultomy of the hip capsular structures, in order to gain better access to the tissue area. Generally reapproximation of the tissue, such as the capsular structures, must be performed with a separate device, which can be expensive and time consuming. Accordingly, a method and device for solving this problem is desired.

### SUMMARY OF THE DISCLOSURE

Described herein are apparatuses and methods for suturing tissue. In particular, described herein are methods and apparatuses for suturing hip (e.g., hip capsule and/or hip labrum) tissue. Any of the apparatuses described herein may include an elongate body and a pair of jaws extending from the distal end of the elongate body that are configured so that a tissue-penetrator (e.g., needle) carrying, or capable of carrying, a suture can be extended between the jaws. Any of these apparatuses may include a sharp/tissue penetrating lower jaw. Any of these apparatuses may be configured so that the lower jaw member can be retracted (partially or completely, including retracting the distal tip) into the elongate body of the apparatus. Any of these apparatuses may also be configured so that either or both the upper and lower jaw are curved (arcuate) towards each other, particularly at their distal ends. For example, any of these apparatuses may include a curved lower jaw that is configured so that a distal end region of the lower jaw curves towards the second jaw. The tissue penetrator may be configured to extend from within the lower jaw and across the distance between the first and second jaws (which may be adjustable) to pass a suture between the two jaws. The upper jaw member may be configured to pivot relative to the elongate body.

For example, described herein are methods of suturing a hip labrum, the method comprising: inserting a suture passer proximate to the hip labrum, the suture passer including an elongate body, a first jaw bent or bendable relative to the elongate body, and a second jaw; positioning the first jaw of the suture passer adjacent the hip labrum; extending a sharp pointed tip of the second jaw through the hip labrum or between the hip labrum and an acetabulum; extending and retracting a tissue penetrator from the first jaw or the second jaw of the suture passer, and through the hip labrum between the first and second jaws to pass a suture between the first and second jaws; and removing the suture passer from the hip labrum while leaving the suture in the hip labrum.

Inserting the suture passer may include minimally invasively inserting the suture passer. In any of the methods of suturing a hip described herein, positioning the first jaw of the suture passer may include positioning the first jaw of the suture passer against the hip labrum. Positioning the first jaw of the suture passer may also or alternatively include adjusting an angle of the first jaw relative to the elongate body such that the first jaw rests against the hip labrum.

Extending the sharp pointed tip of the second jaw may include extending the sharp through the hip labrum. Extending the tissue penetrator from the first jaw or the second jaw of the suture passer may include extending the tissue penetrator from the second jaw. Extending the tissue penetrator from the first jaw or the second jaw of the suture passer may include pushing a suture coupled to the tissue penetrator through the hip labrum. Extending the tissue penetrator comprises may include extending and retracting the tissue penetrator from between the first and second jaws after extending the tissue penetrator. Any of these methods may also include extending the second jaw distally relative to a long axis of the elongate body.

In any of the methods described herein, removing the suture passer may comprise pulling the suture through the hip labrum as the suture passer is removed.

Inserting the suture passer proximate to the hip labrum may comprise inserting a curved first jaw that is bent or bendable relative to the elongate body. The suture passer inserted proximate to the hip labrum may be a curved second jaw that is curved toward the first jaw and includes an arcuate channel therethrough.

Any of the methods of minimally invasively suturing a hip labrum described herein may include: inserting a suture passer proximate to the hip labrum, the suture passer including an elongate body, a first jaw bent or bendable relative to the elongate body, and a second jaw that is distally extendable relative to a long axis of the elongate body; positioning the first jaw of the suture passer against the hip labrum by adjusting an angle of the first jaw relative to the elongate body; extending a sharp pointed tip of the second jaw through the hip labrum or between the hip labrum and an acetabulum; extending and retracting a tissue penetrator from the first jaw or the second jaw of the suture passer, and through the hip labrum between the first and second jaws to pass a suture between the first and second jaws; and removing the suture passer from the hip labrum while leaving the suture in the hip labrum.

Also described herein are suture passers configured for passing a suture that include: an elongate body extending distally and proximally along a long axis; a first jaw extending from a distal end region of the elongate body wherein the first jaw is hinged to pivot and form an angle relative to the long axis at a hinge point with the elongate body; a second jaw having a sharp, tissue penetrating distal tip, wherein the distal end region of the second jaw is curved toward the first jaw and includes an arcuate channel therethrough; and a tissue penetrator configured to carry a suture, the tissue penetrator configured to extend from the arcuate channel and out of the second jaw towards the first jaw at an angle of less than 90 degrees relative to the long axis.

The second jaw may be configured to extend and retract proximally and distally in the long axis relative to the elongate body. In any of the devices describe herein the second jaw may be configured to retract completely into the elongate body.

The first jaw may be configured to have a neutral position in line with the long axis of the elongate body. The first jaw may be straight or may be curved. The first jaw comprises may include a channel for receiving the tissue penetrator. Any of the apparatuses described herein may include a deflection surface on the first jaw configured to deflect the tissue penetrator proximally relative to the first jaw. The first jaw may comprise a suture retainer configured to retain the suture from the tissue penetrator. The first jaw may be configured to retain a tip of the tissue penetrator so that is does not extend laterally beyond the first jaw.

The tissue penetrator may comprise a sharp distal tip comprising a hook region.

For example, described herein are suture passer apparatuses for passing a suture comprising: an elongate body extending distally and proximally along a long axis; a curved first jaw extending from a distal end region of the elongate body, wherein the first jaw is hinged to pivot relative to the long axis of the elongate body; a second jaw having a sharp, tissue penetrating distal tip, wherein the distal end region of the second jaw is curved toward the first jaw and includes an arcuate channel therethrough; a tissue penetrator housed within the second jaw and configured to carry a suture, the tissue penetrator configured to extend from the arcuate channel and out of the second jaw towards the first jaw at an angle of less than 90 degrees relative to the long axis; a handle at the proximal end of the elongate body having a control for controlling pivoting of the curved first jaw; and a deflection surface on the first jaw configured to deflect the tissue penetrator proximally relative to the first jaw.

Also described herein are methods of re-approximating tissue using a suture passer having an elongate body, a first jaw, and a second jaw. For example, the method may include: positioning the suture passer proximate to (e.g., next to, adjacent, immediately adjacent) a first tissue; piercing the first tissue with a sharp tip of the second jaw of the suture passer; extending an retracting a tissue penetrator through the second tissue between the first and second jaws to pass a suture between the first and second jaws while the first tissue is pierced by the second jaw; and pulling the second jaw out of the first tissue while leaving the suture in the first tissue and second tissue.

As mentioned above, the step of positioning may be performed minimally invasively.

In some variations, the method includes the step of positioning the second jaw adjacent to the second tissue (e.g., before passing the needle/tissue penetrator). For example, the suture passer may be used to position the first tissue near the second tissue by pulling the first tissue while on the second jaw of the suture passer near the second tissue. This step may include piercing a second tissue with the sharp tip of the second jaw.

Thus, any of the methods described herein may also include pulling the pierced first tissue towards the second tissue with the suture passer. Any of these methods may also include adjusting the angle of the first jaw relative to the elongate body by pivoting the first jaw relative to the elongate body.

Any of these methods may also include the step of sliding the second jaw from the elongate body to extend the sharp pointed tip.

As described above, the methods of re-approximating tissue described herein may be, in particular, applied to repair of hip, such as hip labrum and/or hip capsule. For example, the first tissue may comprise a first region of hip capsule and the second tissue may comprise a second region of hip capsule.

Pulling the second jaw out of the second tissue and the first tissue may comprise withdrawing the second jaw into the elongate body to retract the sharp pointed tip. Piercing the first tissue with the sharp tip of the second jaw may comprise piercing the first tissue with a curved distal end region of the second jaw.

For example, a method of minimally invasively re-approximating tissue using a suture passer having an elongate body, a first jaw, and a second jaw may include: positioning the suture passer proximate to a first tissue; piercing the first tissue with a sharp tip of the second jaw of the suture passer; pulling the pierced first tissue towards a second tissue with the suture passer; positioning the second jaw immediately adjacent or next to the second tissue (in some variations, piercing the second tissue with the sharp tip of the second jaw); adjusting the angle of the first jaw relative to the elongate body; extending an retracting a tissue penetrator through the second tissue between the first and second jaws to pass a suture between the first and second jaws while the first tissue is pierced by the second jaw; and pulling the second jaw out of the second tissue and the first tissue while leaving the suture in the first tissue and second tissue.

### BRIEF DESCRIPTION OF THE DRAWINGS

FIG. 1A shows a representation of a typical hip labrum.

FIG. 1B shows a hip labrum with chrondo-labral separation.

FIG. 2A is one embodiment of an exemplary suture passer device as described herein having a sharp (tissue penetrating) and slideable lower jaw, and a pivoting upper jaw; the lower jaw may be retracted/extended.

FIG. 2B is another embodiment of an exemplary suture passer device as described herein. In FIG. 2B, the lower jaw may be fixed relative to the elongate body, and does not extend/retract, but is sharp/tissue penetrating.

FIGS. 3A-3M illustrate the use of a suture passer device such as the one shown in FIG. 2A to repair a hip labrum.

FIGS. 4A and 4B show another embodiment of an exemplary suture passer device.

FIGS. 4C, 4D and 4E illustrate the extension of a second needle (tissue penetrator) configured to couple with a suture from the lower jaw, where the lower jaw, such as the lower jaws shown in FIGS. 4A and 4B, is also configured to penetrate tissue (as a first needle).

FIGS. 5A-5L illustrate use of a suture passer device such as the one shown in FIGS. 4A and 4B to repair a hip labrum.

FIG. 6 shows an exemplary stitch to repair a hip labrum.

FIGS. 7A-7E illustrate the use of a suture passer to simultaneously position and suture tissue; in FIGS. 7A-7E, the method and apparatus is illustrated for re-approximating and repairing a hip capsular structure.

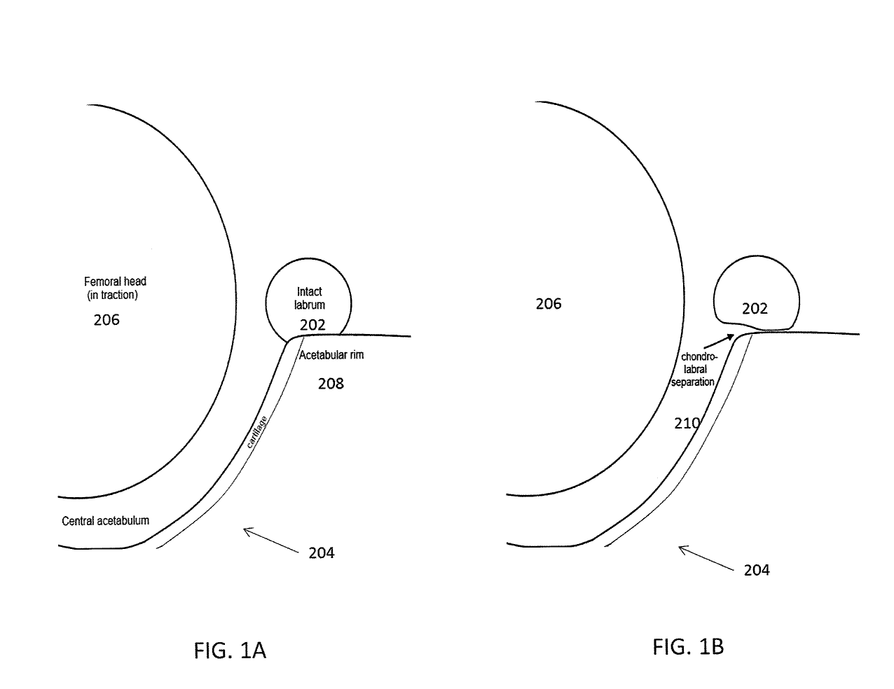

### DETAILED DESCRIPTION

Described herein are devices and methods for suturing tissue, and particularly suture passer devices for re-approximating and suturing tissue, such as hip labrum.

In general, the suture passers described herein may be used as a low-profile suture passer that can be minimally invasively (e.g., arthroscopically) inserted into even very tight, congested, or narrow regions, or any other difficult-to-reach portion of the body, such as the hip labrum; the suture passer may be positioned around a target tissue to pass a suture from one side of the tissue to another side. The suture passers described herein may also be used to manipulate the tissue before or during suture passing, so that the tissue is positioned within the body as desired, and held in alignment and/or position for suturing using the same device used to pass the suture. The suture passer devices described herein may be referred to as suture passers and/or suturing devices.

In general, the suture passers described herein include a first jaw member and second jaw member that extend from the end of an elongate body region to form a distal-facing mouth into which tissue to be sutured fits. In some variations, one or both jaws forming the mouth may be independently moved. The first jaw member may be referred to as an “upper” or bending jaw member, and the second jaw member may be referred to as a lower or sliding jaw member; the first and second labels may be reversed, as indicated by the context.

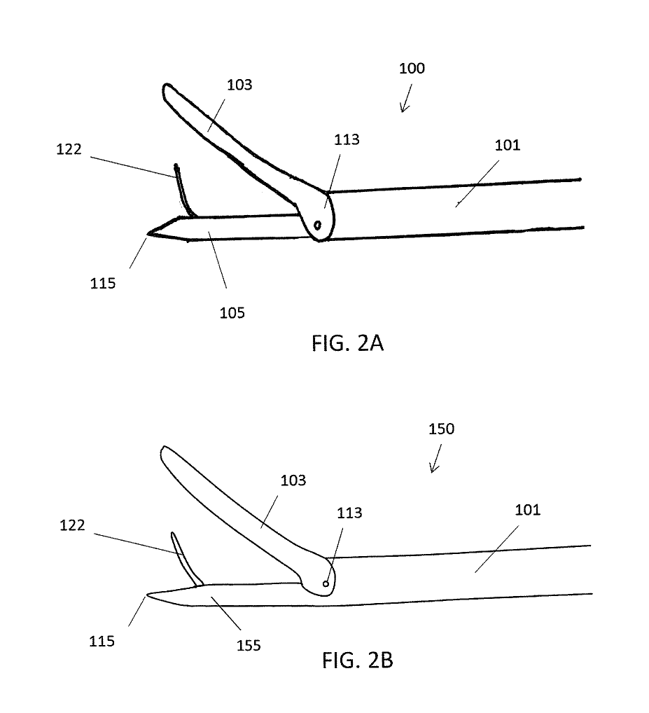

FIG. 2A illustrates one variation of a suture passer 100. In this example, the device has a first (upper) jaw member 103 extending distally from the distal end of a more proximal elongate member 101. A second jaw member 105 is shown extended distally beneath the first jaw member 103. This lower 105 jaw member may be retracted or extended from the elongate body, and has a tissue-penetrating (e.g., sharp) distal end 115. Further, a needle (“tissue penetrator”) 122, shown here as a flexible needle, extends from the second jaw member 105. A handle (not shown) can be located at the proximal end of the device and can include multiple controls for independently controlling the movements of the first jaw member, second jaw member, and/or tissue penetrator.

As mentioned, the second jaw member 105 includes a pointed or sharp tip 115 configured to penetrate into tissue and/or penetrate through narrow passageways between tissue, cartilage, or other obstacles within the body. The sharp tip 115 can be substantially aligned with the elongate member 101 so as to penetrate in a distal direction. As described further below, in some embodiments, the second jaw member 105 can be retractable into the elongate member 101.

The suture passer 100 in FIG. 2A is positioned with the first jaw member 103 held at an angle relative to the long axis of the proximal elongate member 101. The first jaw member 103 in this example is shown having a hinge region 113 about which the first jaw member 103 may be angled relative to the elongate member 101. In some variations, this hinge region 113 is a pinned hinge. In other embodiments, non-pinned (e.g., living hinges) regions may be used. Any appropriate articulating region that allows the first jaw member to move at an angle relative to the proximal portion of the device (e.g., the elongate member) may be used. In some variations, this first jaw member 103 is referred to as an upper jaw member, but alternative variations (in which the first jaw member is a lower jaw member) are also possible. The hinged jaw member may be biased either open, closed, or some intermediate position (e.g., 10 degrees, 15 degrees, 20 degrees, 25 degrees, 30 degrees, etc.).

The first jaw member and second jaw member may be actuated by any appropriate mechanism, including a tendon member (e.g., push rod, pull rod, or the like), and may be held (locked) at any angle (e.g., between 0° and 180° relative to a line extending from the distal end of the elongate body, between about 0° and 90°, between about 0° and 60°, etc.). In some variations, the device has a neutral position during which no force is applied to the controller to move the first jaw member, so that the first jaw member is angled “open” (e.g., at 30°, 45°, 50°, 90° or at any angle between about 15° and about 90°) relative to the elongate body; actuating the control on the handle results in the first jaw member moving towards the “closed” position (e.g., reducing the angle with respect to a line extending from the distal end of the elongate body). In some variations, the jaw member is in the neutral position when angled with 0°/180° relative to the elongate body.

The first jaw member 103 shown in FIG. 2A also includes a suture retainer region near the distal end. This suture retainer region may hold a suture or be configured to hold a suture. In some variations, the suture retainer includes a channel or guide for holding the suture in a preferred position. In some variations the suture retainer includes a pair of graspers, or deflectable members into which the suture may be pushed and held (e.g., handed off from the tissue penetrator). A suture retainer generally holds the suture so that it can be either removed by the tissue penetrator, or so that a suture can be passed into the suture retainer from the tissue penetrator. A suture may be wrapped around the first jaw member 103 to create a loop of suture within the first jaw member 103. This loop of suture held by the suture engagement region of the jaw member 103 may be held under sufficient tension so that the suture may be engaged by the suture engagement region of the tissue penetrator 122. In some variations, a tensioning member may be included in the suture engagement region. In some variations, the suture may be contained within the elongate body of the device. Alternatively, the suture may be kept outside of the device. In some variations, the suture may be loaded by the user. For example, a user may load a suture on the device by placing a loop of suture over the first jaw member. In some variations, the suture holder may be placed along the length of the device to hold or manage the suture so that it doesn't interfere with the operation of the device or get tangled.

The lower jaw member 105 can be configured to slide proximally towards and into the proximal elongate body 101 of the device 100. The second jaw member 105 can thus move axially in the direction of the proximal-distal axis of the suture passer. In some variations the second jaw member 105 moves axially completely past the distal end of the elongate body. Alternatively, the second jaw member 105 slides axially in the proximal direction only partially (e.g. to align with the hinge region of the first jaw member). The second jaw member 105 shown in FIG. 2 retracts completely into, and extends out of, the lower portion of the elongate body 101.

The tissue penetrator 122 may be housed within either the first or second jaw member. The tissue penetrator 122 may be configured as a needle, wire, knife, blade, or other element that is configured to extend from within either the first or second jaw members and across the opening between the jaw members to engage a suture retainer and either drop off or pick up a suture therefrom. In general, the tissue penetrator 122 may be configured to completely retract into the jaw member housing, such as the second jaw member 105. It may be extended across the opening between the jaw member 103, 105 by actuating a member in the handle to push or otherwise drive it across the opening and through any tissue held between the jaw members 103, 105.

The elongate body 101 shown in FIG. 2A is illustrated as a relatively straight cylindrical body, though other shapes may be used. For example, the elongate body 101 may be curved, bent, or angled. In some variations, the elongate body is configured to be bent, curved or angled dynamically (e.g. by changing the bend or curve). Further, the elongate body 101 may be any appropriate length. For example, the elongate body may be between about 6 and about 24 inches long, e.g., 6 inches long, 8 inches long, 10 inches long, 12 inches long, etc. The suture passers described herein may be used for arthroscopic surgeries and therefore may be dimensioned for use as such. Thus the diameter of the device 100 may be configured to be small enough for insertion into a cannula, tube or the like for insertion into the body.

In some variations, the lower jaw may be fixed or non-slideable, as illustrated in FIG. 2B. In this variation, the suture passer 150 includes a hinged upper jaw 103, which is hinged at a pivot point 113, as discussed above for FIG. 2A. However, in this example, the lower jaw member 155 is an extension of the elongate body 101 and is not retractable relative to the elongate body 101. The device may otherwise share any of the features described above. As in FIG. 2A, the tissue penetrator (needle 122) is extendable and retractable into the lower jaw member (partially or completely), and the lower jaw is also tissue penetrating.

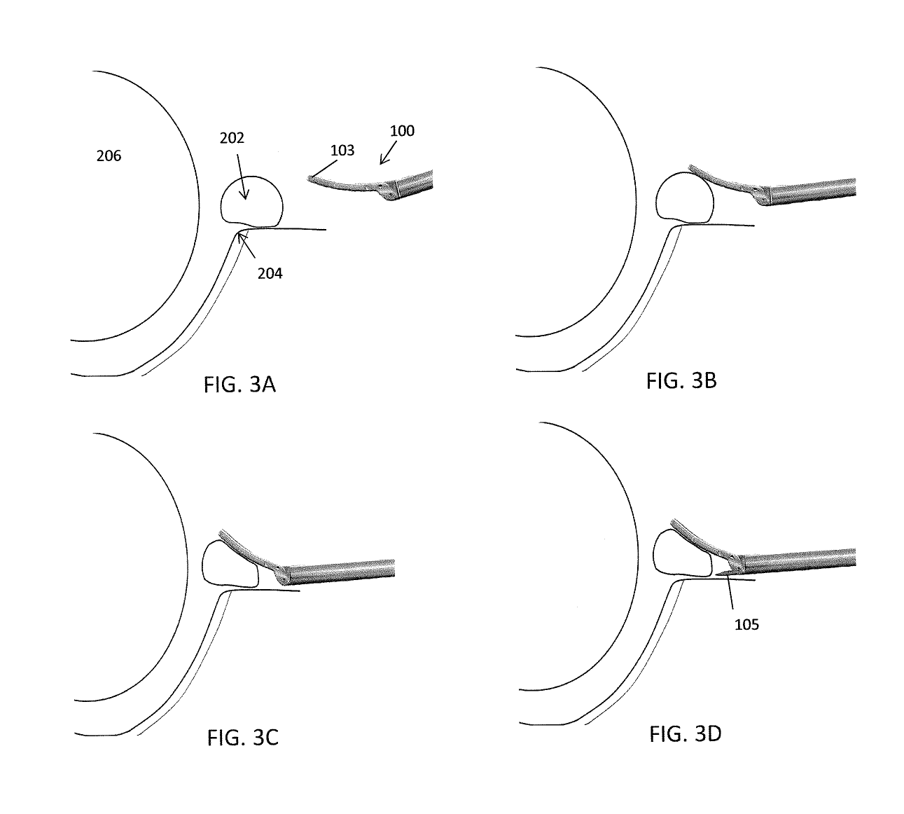

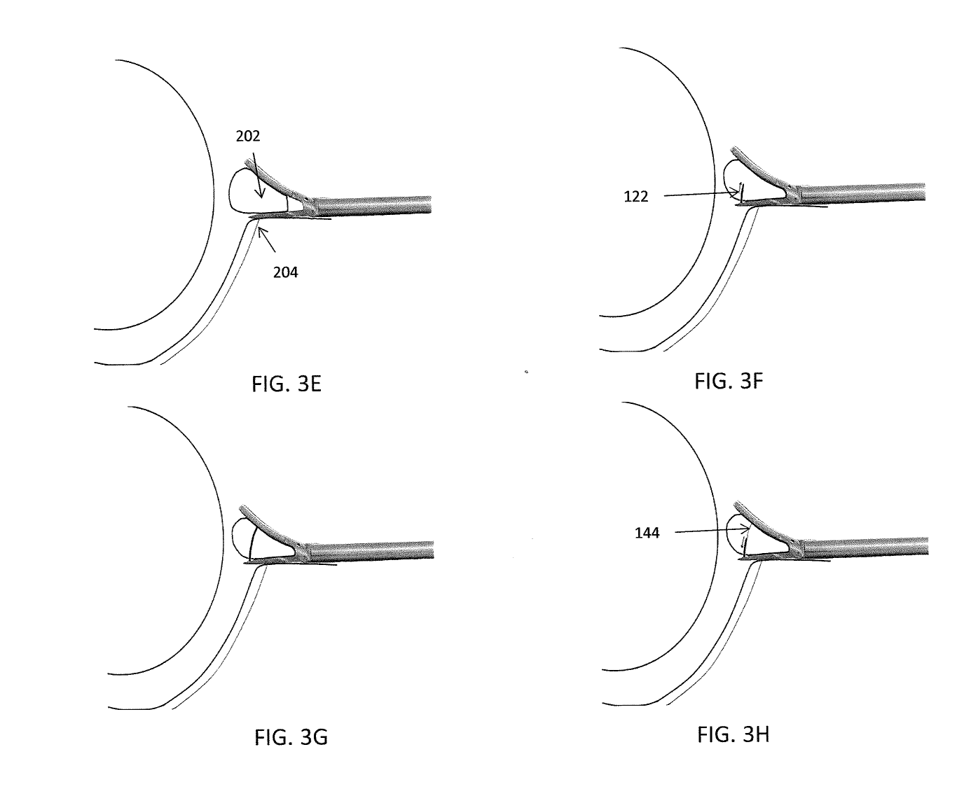

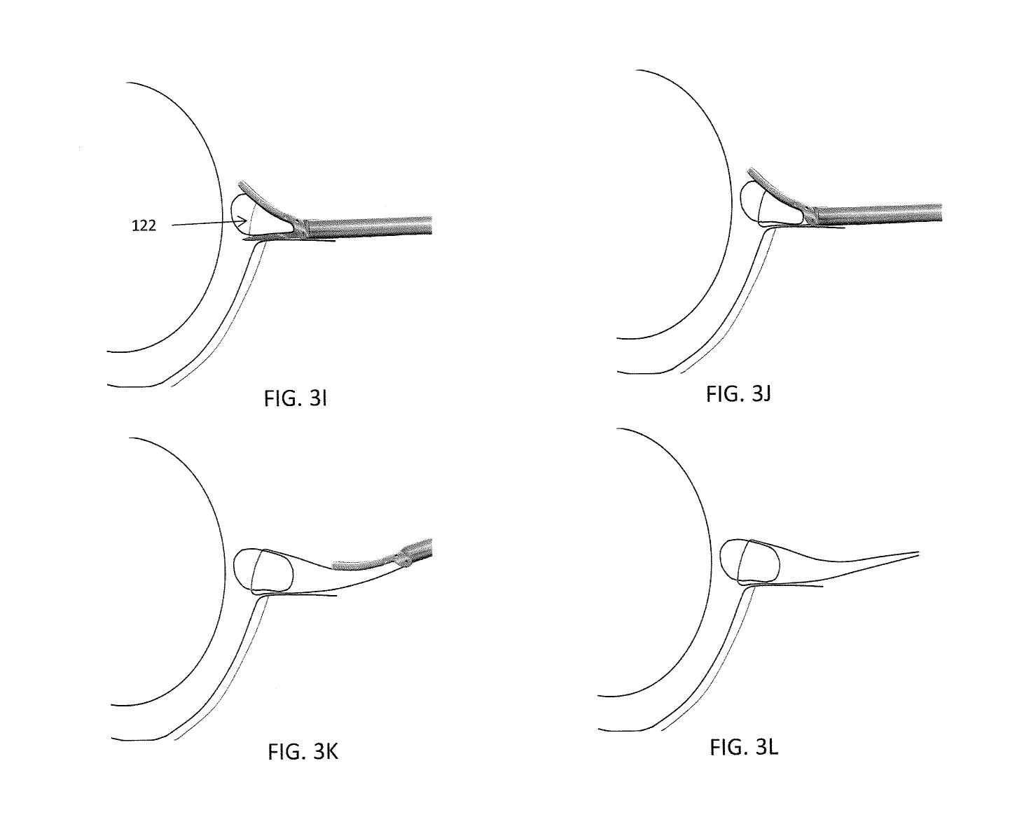

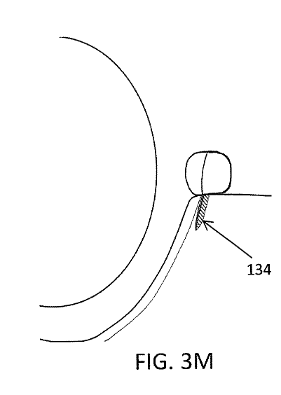

In general, the suture passer devices 100, 150 described herein may be used to suture any appropriate tissue. The device 100 is particularly well suited for passing a suture in a minimally invasive procedure to reach difficult to access regions, such as the hip labrum. An example of the use of the device 100 in the hip labrum is illustrated in FIGS. 3A to 3M.

Before use, a suture 144 (not visible in FIGS. 3A-3G) may be loaded on the first jaw member 103 of the device 100. For example, a loop of suture may be loaded onto the first jaw member. The free ends of the suture may be coupled to a suture control element, such as a tensioning screw or other portion of the suture passer. For example, the two free ends may be cinched onto a tensioner screw. The suture passer 100 may be loaded outside of the body by the user, or it may be pre-loaded. Once loaded, the suture passer 100 may be inserted into the body near the target hip labrum 202. For example, the device may be inserted into the body through a cannula. As shown in FIG. 3A, the second (lower) jaw 105 member may be fully retracted proximally, and the upper jaw 103 may be clamped down fully so that it is in-line with (or approximately straight) relative to the elongate member 101; the first jaw member 103 may be locked in this position for insertion, or it may be moved or dynamically adjusted as it is inserted.

Thereafter, the device may be positioned relative to the hip labrum 202. For example, the first jaw member 103 position may be positioned such that it is held on one side of the labrum (e.g. a labrum that has separated from an acetabular rim, as shown in FIGS. 3A-3L). Thereafter, the lower jaw member may be extended into the tissue between the labrum 202 and the acetabular rim 204

For example, as shown in FIGS. 3B-3D, once the first jaw member 103 is positioned as desired relative to the hip labrum 202, the second jaw member 105 may be extended toward the space between the labrum 202 and the acetabulum 204 formed due to chrondo-lateral separation, as shown in FIG. 3D. The sharp edge 115 of the second jaw member 105 can extend between the acetabular rim and the labrum tissue, as shown in FIG. 3E. In this example, the control for the second jaw member (the lower jaw lock) may be actuated to slide the lower jaw member distally, forming the distal-facing opening, and surrounding (at least partially) the hip labrum 202. Alternatively, in some variations a fixed, non-sliding lower jaw may be used, and may be positioned between the tissues before placing the upper jaw alongside the labrum (not shown).

Referring to FIGS. 3F and 3G, thereafter, the needle/tissue penetrator 122 may be actuated (e.g., by squeezing a needle trigger) to extend from within the lower jaw member 105, through the hip labrum 202, between the first and second lower jaw members 103, 105, and across to the upper jaw 103. As mentioned, the suture may be pre-loaded, either in the lower jaw (and thereby passed as the needle extends from the lower jaw, as shown in FIG. 3F) or in the upper jaw 103. If the loop of suture is held in the upper jaw, the needle may to engage the suture held within the suture engagement region in the upper jaw 103 and pull it back down through the tissue as the needle is retracted back to the lower jaw; alternatively, the suture 144 may be preloaded in the lower jaw and pushed with the needle through the labrum as the needle extends from the lower jaw to the upper jaw, as shown in FIG. 3F; the suture 144 may then be held in the upper jaw while the needle is retracted back to the lower jaw, as illustrated in FIGS. 3H-3I.

Once the suture has been passed between the jaws (as shown in FIG. 3I), the tissue penetrator may be fully retracted back into the lower jaw member 105. Thereafter the lower jaw member may be retracted into the elongate body, as shown in 3J, proximally. The entire suture passer may then be withdrawn from the tissue (as shown in FIG. 3K-3L), in the reverse to the process described above, so that the suture passer 100, which having passed the suture 144 successfully, is withdrawn from the patient, leaving a loop of suture passed through the labrum and between the labrum and the acetabular rim, as shown in FIG. 3L. Once the suture 144 has been placed, it can be used to secure the labrum fully down on the acetabular rim, and knotted or tied down with a suture anchor 134, as shown in FIG. 3M.

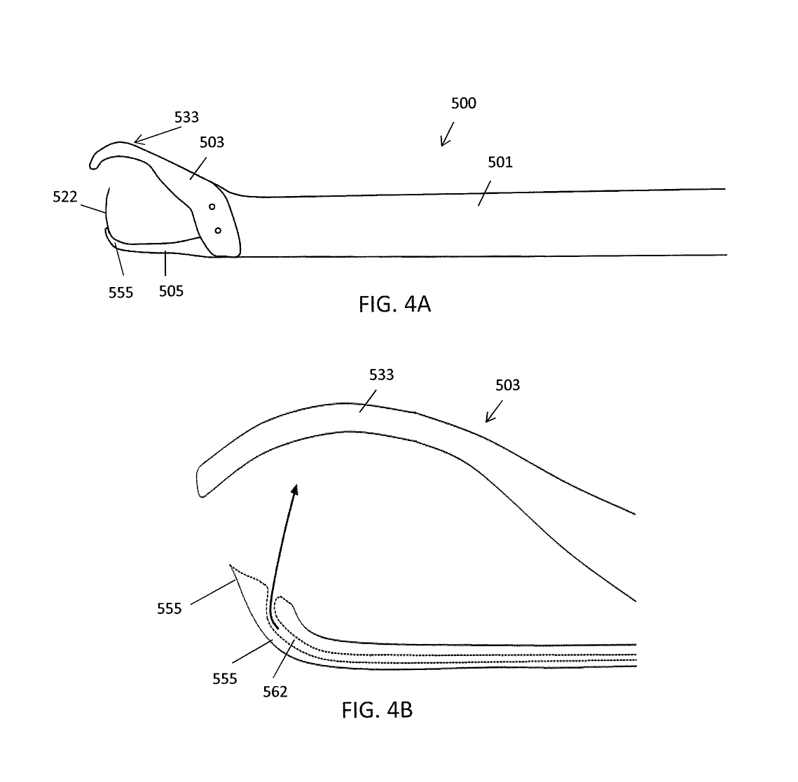

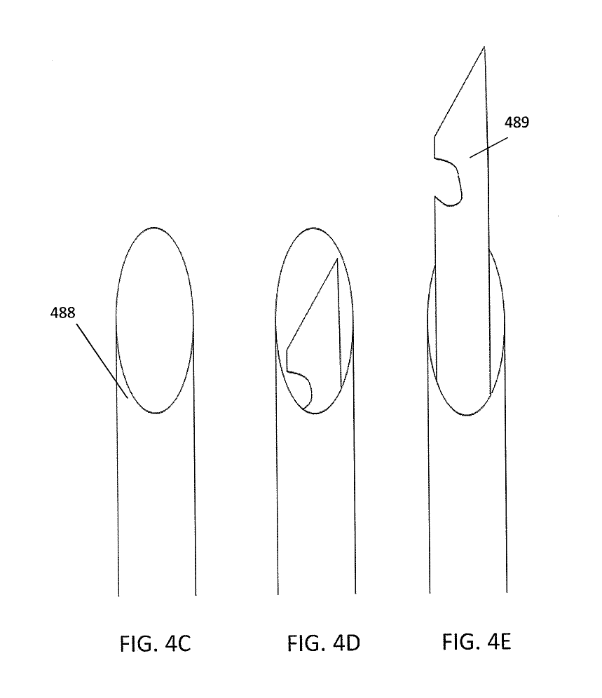

Some suture passers described herein may include curved upper and/or lower jaws, which may help in positioning and manipulating the tissue to be sutured. Referring to FIGS. 4A and 4B, a suture passer 500 can include similar features to the suture passer 100 described above, such as an elongate body, two jaws, a sharp pointed edge on the lower jaw 515, a bendable upper jaw, retraction of the lower jaw, etc. However, in this embodiment, either or both the upper and/or lower jaws include arcuate or bowed segments 533, 555 that may make it easier to grab and/or hold a piece of tissue, such as the hip labrum, therebetween. That is, the arcuate segments 533, 555 can bow away from one another to thereby make a rounded capturing space therebetwen. In FIGS. 4A and 4B both the lower and upper jaws are shown with curved distal end regions; it should be understood that either the lower jaw alone or the upper jaw alone may be curved as illustrated.

The lower jaw 503 of the suture passer 103 can include both a tissue penetrator 522 and/or in some variations a suture retainer. As shown in FIG. 4B, the lower jaw 505 can include an arcuate passage 562 for the tissue penetrator 522 that extends through the jaw 505 and then angles slightly backwards or proximally. Accordingly, as a tissue penetrator 522 extends through the lower jaw 505 and the arcuate passage 562, it can push/pull the suture 544. As the tissue penetrator 522 extends through the passage and out into the tissue, it may extend from the lower jaw 505 at an angle (e.g., an angle of less than 90 degrees, less than 85 degrees, etc.,) relative to the longitudinal axis of the device 500 and/or the elongate member 501, as shown by the arrow in FIG. 4B.

The upper jaw 503 can include a suture capture mechanism (not shown) configured to house the suture passed by the tissue penetrator after it has passed into the upper jaw 103. In some variations, the upper jaw may be configured to deflect the tissue penetrator so that it is directed proximally (e.g., towards the handle) by including a channel (not shown) in the upper jaw. After the tissue penetrator 522 has passed (with the suture 544) from the lower jaw 505, the suture 544 may be held by the capture mechanism in the upper jaws, such as a displaceable spring leaf or biased plate (that can be displaced by the tissue penetrator). This capture mechanism can be positioned at or proximal to entry point of the penetrator 522 into the upper jaw 503. Thus, the tissue penetrator 522 can be deflected within the upper jaw 503 and allowed to extend proximally through the opposite jaw member a set amount (e.g., less than 5 mm, about 5 mm, less than 4 mm, about 4 mm, etc.) until the suture 544 is captured by the suture capture mechanism.

Having the tissue penetrator 522 extend proximally within the upper jaw 503 (i.e. into the capture mechanism 560) can be advantageous for several reasons. For example, less coordination is required to limit the needle motion (e.g., stopping it before it crashes into the first or upper jaw). This may allow greater tolerances, and the parts may require less precision. Also, extending the tissue penetrator 522 proximally may prevent damage to adjacent tissues, and may allow for “over travel” of the tissue penetrator and provide for more reliable engagement (hooking) of the suture by the suture engagement region. The first jaw member may 503 include sufficient space for the tissue penetrator to over-travel the suture so that the hook (suture engagement feature) on the tissue penetrator can grab the suture on its way back to the lower (second) jaw member in variations in which the upper jaw member is preloaded with suture. With this variation, the length of the first jaw member 503 can be reduced, thereby avoiding disruption by an overhang of the first jaw member 503 that can occur in tissue in tight spaces.

A suture may be freely movable relative to the lower jaw 503. Accordingly, once the suture is fixed in the capture mechanism 560, relative movement of the device 500 can result in leaving the suture in the desired location of the tissue.

FIGS. 4C to 4E illustrate one example of a lower jaw member 488 that houses a needle, tissue penetrator 489. The needle may be fully retracted into the lower jaw.

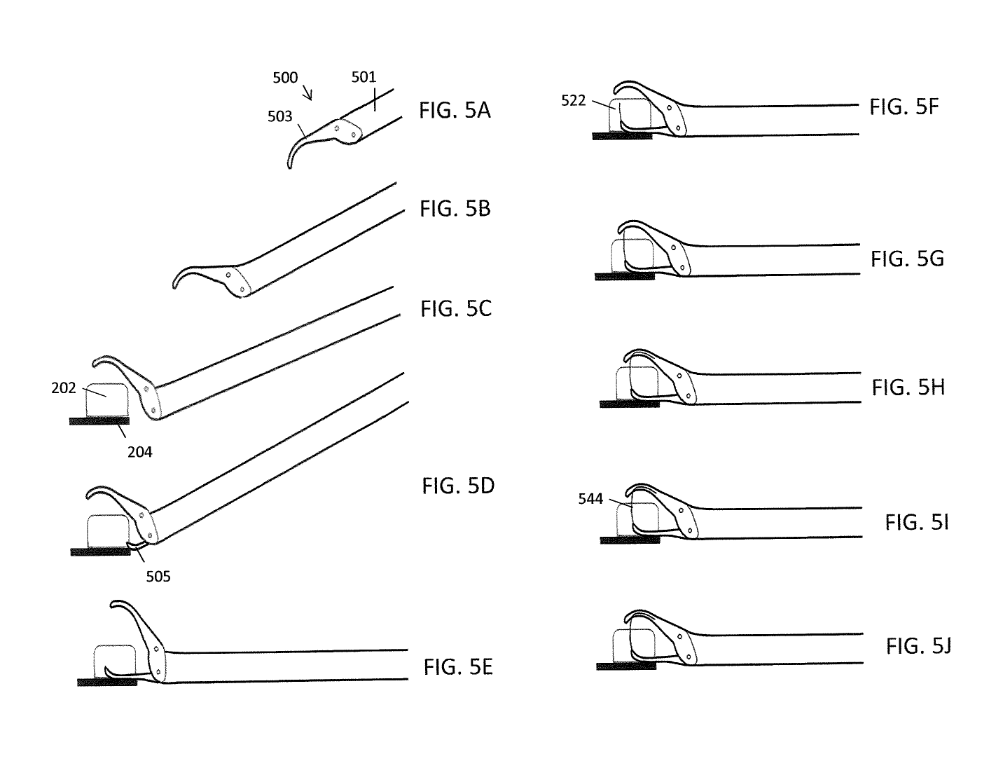

Any of the devices described herein may be particularly useful for manipulating (positioning, holding, etc.) one or more pieces of tissue to be sutured. In general, a suture passer device 500 as described herein may be used to suture any appropriate tissue. As described above, these devices 100 are particularly well suited for passing a suture in a minimally invasive procedure to reach difficult to access regions, such as the hip labrum. An example of the use of the device 500 in the hip labrum is illustrated in FIGS. 5A to 5L.

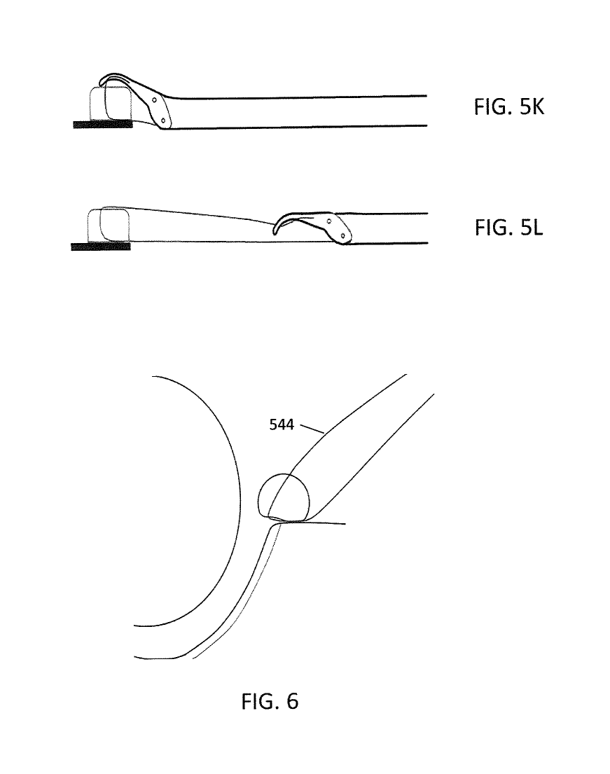

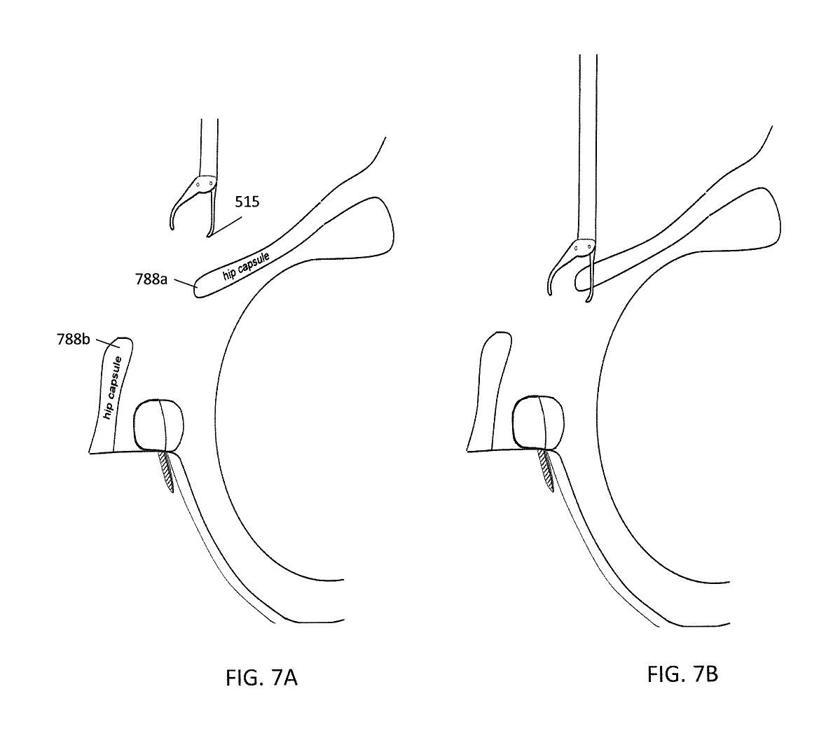

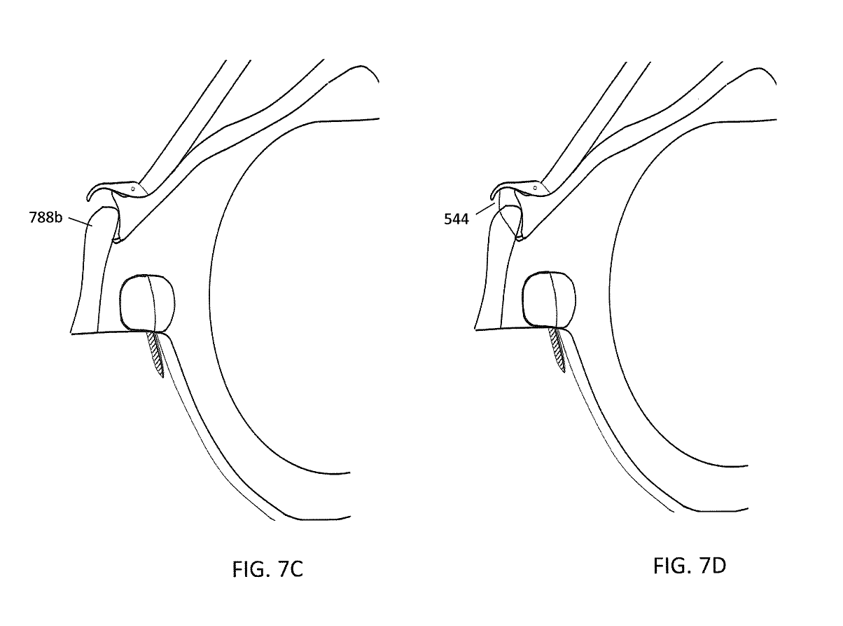

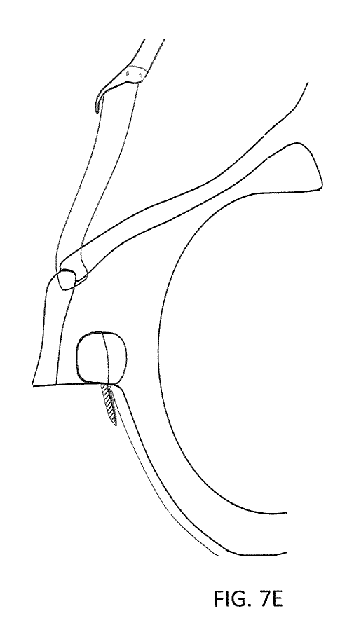

Referring to FIGS. 5A and 5B, a suture passer 500 can be moved into the body until it reaches the hip labrum 202. FIGS. 5A and 5B show the suture passer 500 approaching a hip labrum tissue (within the body). The orientation of the upper jaw 503 relative to the elongate member 501 can then be adjusted such that the upper jaw 503 is able to sit against the top of the hip labrum 202, as shown in FIG. 5C. Referring to FIG. 5D, the lower jaw 505 can then be extended between the hip labrum and the acetabular rim 204. In some variations, this may include placing the lower jaw into a gap between the two formed as a result of the chrondo-lateral separation. As the lower jaw 505 is extended further, the sharp tip of the lower jaw 505 can pierce through the labrum 202, as shown in FIG. 5E. In FIG. 5F the tissue penetrator (needle) 522 can then be extended from the lower jaw 505. The arcuate portions 533, 555 of the upper and lower jaws 503, 505 may aid the upper and lower jaws 503, 505 to surround the labrum 202 therebetween. Finally, the arcuate portion 533 and the arcuate passage of the lower jaw member 503 may allow the tissue penetrator 522 to extend though the labrum to the upper jaw (as shown in FIGS. 5F-5G). The tissue penetrator 522, once it reaches the upper jaw 503, can extend proximally into the upper jaw 503 (as shown in FIGS. 5H-5J), leaving the suture loop passed from the lower jaw in the upper jaw capture region (not shown). Once the suture 544 is held in the upper jaw, the penetrator 522 can be retreated into the lower jaw 505, the lower jaw 505 can be retracted (as shown in FIG. 5K), and the entire device 500 can be pulled distally to thereby pull the suture 544 through the tissue. Once the loop of suture has been passed, the suture may be secured to the bone (e.g., acetabular region) to hold the labrum securely against the ascetabulum, as described above. FIG. 6 shows the suture passed through the labrum and partially around it, between the labrum and the acetabular rim. This stitch may be tightened around the labrum and anchored. An anchored labrum repaired as described above is shown in FIGS. 7A-7E, which also illustrate the operation of the device to both manipulate and suture (e.g., repair) other tissues, including a torn or separated hip capsule.

For example, a suture passer device can also advantageously be used to repair tissue cut during the initial suturing procedure. In FIGS. 7A-7E, the suture passer device 500 can be used to reapproximate two pieces 777a, 777b of hip capsular tissue. That is, referring to FIGS. 7A-7B, the sharp tip 515 of the lower jaw can be used to pierce first piece 777a of hip capsular tissue, as shown in FIG. 7B. The entire device 500 can then be pulled towards the second piece 777b of hip capsular tissue. In some variations the sharp tip 515 may also pierce the second piece 777b of capsular tissue, as shown in FIG. 7C. In any event, the suture passer, having hooked at least one piece of tissue, may position the tissue so that the two pieces 777a,777b of tissue will then be proximate to one another on the lower jaw 505. As shown in FIG. 7D, the needle (e.g., tissue penetrator) can then pass the suture 544 from the lower jaw 505, through the tissue 777b, and into the upper jaw 503. The needle and lower jaw 505 can then be retracted. Finally, the entire device 500 pulled proximally to leave the suture 544 such that it binds the two pieces 777a,b together, as shown in FIG. 7E. Because the two pieces are joined as using a single pass of the device, this may prevent tissue bridging and girth hitches commonly encountered with capsule repair techniques.

The devices described here may include a retractable lower jaw, as shown, although the lower jaw need not be retractable. Further, in some embodiments, the upper jaw can be flexed down over the sharp tip of the lower jaw 105, 505 to protect the tip during insertion and removal of the device.

In general, the sutures passers described herein may be used arthroscopically, and may be used to pass one or more length of suture. As mentioned above, the suture passers described herein may include an elongate body and a first jaw member (e.g., first jaw) extending from the distal end of the elongate body, wherein the first jaw is bent or bendable relative to the distal to proximal axis of the elongate body. In some variations the first jaw is hinged near the distal end region of the elongate body. Some variations of the suture passers described herein include a second jaw member (e.g., second jaw) that is configured to slide axially (proximally and distally) relative to the elongate body and/or first jaw. The first and second jaws may be configured to form a distal-facing opening into which tissue may be held. The suture passers described herein may also include a flexible, bendable, or pre-bent tissue penetrator for passing a suture through the tissue. The suture passer may also include a handle at the proximal end with one or more controls for actuating the first and/or second jaws and the tissue penetrator.

In particular, the devices described herein, which may be particularly well adapted to perform these methods, may include an axially slideable second jaw that is configured to penetrate tissue. This configuration may allow the device to pass the suture in an angled pathway through the tissue, including “L-shaped” pathways within the tissue, when extending the tissue penetrator.

The lower (tissue penetrating) jaw may be particularly well adapted by including a small profile while still carrying and/or guiding positioning of the tissue penetrator (“needle”) carrying the suture. For example, in some variations, described herein are suture passer having very narrow second jaws; the tissue penetrator may exit the second jaw from the side of the second jaw and extend across a distal-facing opening to engage an opening in the opposite jaw (e.g., the first jaw), where a suture may be secured and/or released. A suture passer may have a second jaw having a maximum diameter (e.g., maximum height) along the length of the second jaw of less than about 0.11 inches, 0.10 inches, 0.09 inches, 0.08 inches, 0.07 inches, 0.06 inches, 0.05 inches, 0.04 inches, 0.03 inches, 0.2 inches, 0.01 inches, etc. The second jaw may be any appropriate width. For example, the width may be approximately 0.15 inches, less than 0.15 inches, less than 0.14 inches, less than 0.13 inches, less than 0.12 inches, less than 0.11 inches, less than 0.10 inches, less than 0.09 inches, less than 0.08 inches, less than 0.07 inches, less than 0.06 inches, less than 0.05 inches, etc. These dimensions may refer to the maximum high, width, breadth, or thickness of the lower jaw (e.g., in particular along the distal end region, such as the distal-most 3 cm, 5 cm, 6 cm, 7 cm, 8 cm, 9 cm, 10 cm, etc.). In general, the tissue penetrator (needle) may be held within and/or on top of the lower jaw. Thus the tissue penetrator may be the same width as the lower jaw, lightly larger, or slightly smaller than the lower jaw.

In some variations, described herein are suture passers that do not include a second (e.g., lower) jaw, but that are instead configured so that a tissue penetrator (e.g., needle, ribbon, etc.) extend from the distal end region of the elongate member to engage a side region of the first jaw to pass a suture through the tissue. An elongate member may be any elongate structure extending from the proximal to distal end region of the device (e.g., cannula, tube, cylinder, arm, shaft, etc.).

In some variations, the issue penetrator extends or travels from the second (e.g., lower) jaw to the first (e.g., upper) jaw in a sigmoidal (e.g., approximately “S-shaped”) path. One or more length of a suture (including two lengths of the same suture, e.g., two ends of the same suture) can be loaded into the second jaw and/or tissue penetrator and passed from the second jaw, through the tissue and retained in the first jaw, to pass a length of suture through the tissue. Thus, for example, the tissue penetrator may be deflected when exiting or extending from the lower (second) jaw and after contacting the upper (first) jaw. In variations including a lower jaw that is very low profile (e.g., having a width of less than about 0.07 inches), tissue penetrator may be pre-biased to curve up towards the upper jaw when extending from the lower jaw (e.g., when leaving the lower jaw in variations in which the tissue penetrator is held within the lower jaw). In some variations the lower jaw does not include a ramp or deflection region which may allow the lower jaw to be thinner along the entire distal end region.

The suture passer may also be configured so that the first (e.g., upper) jaw can pivot to assume a different angle relative to the elongate body of the device, and the second jaw is axially slideable or extendable distally from the distal end of the elongate member to form a distal-facing mouth with the first jaw. The proximal handle includes a plurality of controls for controlling the pivoting of the first jaw, the axial sliding of the second jaw, and the extension/retraction of the tissue penetrator from the second jaw.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of suturing a hip labrum, the method comprising:
inserting a suture passer proximate to the hip labrum, the suture passer including an elongate body, a first jaw bent or bendable relative to the elongate body, and a second jaw;
positioning the first jaw of the suture passer adjacent the hip labrum;
extending a sharp pointed tip of the second jaw through the hip labrum or between the hip labrum and an acetabulum;
extending and retracting a tissue penetrator from the first jaw or the second jaw of the suture passer, and through the hip labrum between the first and second jaws to pass a suture between the first and second jaws; and
removing the suture passer from the hip labrum while leaving the suture in the hip labrum.

2. The method of claim 1, wherein inserting the suture passer comprises minimally invasively inserting the suture passer.

3. The method of claim 1, wherein positioning the first jaw of the suture passer comprises positioning the first jaw of the suture passer against the hip labrum.

4. The method of claim 1, wherein positioning the first jaw of the suture passer comprises adjusting an angle of the first jaw relative to the elongate body such that the first jaw rests against the hip labrum.

5. The method of claim 1, wherein extending the sharp pointed tip of the second jaw comprises extending the sharp through the hip labrum.

6. The method of claim 1, wherein extending the tissue penetrator from the first jaw or the second jaw of the suture passer comprises extending the tissue penetrator from the second jaw.

7. The method of claim 1, wherein extending the tissue penetrator from the first jaw or the second jaw of the suture passer comprises pushing a suture coupled to the tissue penetrator through the hip labrum.

8. The method of claim 1, wherein extending the tissue penetrator comprises extending and retracting the tissue penetrator from between the first and second jaws after extending the tissue penetrator.

9. The method of claim 1, further comprising extending the second jaw distally relative to a long axis of the elongate body.

10. The method of claim 1, wherein removing the suture passer comprises pulling the suture through the hip labrum as the suture passer is removed.

11. The method of claim 1, wherein the suture passer inserted proximate to the hip labrum comprises a curved first jaw that is bent or bendable relative to the elongate body.

12. The method of claim 1, wherein the suture passer inserted proximate to the hip labrum comprises a curved second jaw that is curved toward the first jaw and includes an arcuate channel therethrough.

13. A method of minimally invasively suturing a hip labrum, the method comprising:
inserting a suture passer proximate to the hip labrum, the suture passer including an elongate body, a first jaw bent or bendable relative to the elongate body, and a second jaw that is distally extendable relative to a long axis of the elongate body, wherein a distal end of the second jaw is curved towards the first jaw;
positioning the first jaw of the suture passer against the hip labrum by adjusting an angle of the first jaw relative to the elongate body;
extending a sharp pointed tip of the second jaw through the hip labrum or between the hip labrum and an acetabulum;
extending and retracting a tissue penetrator from the first jaw or the second jaw of the suture passer, and through the hip labrum between the first and second jaws to pass a suture between the first and second jaws; and
removing the suture passer from the hip labrum while leaving the suture in the hip labrum.

14. A method of minimally invasively suturing a soft tissue in a joint, the method comprising:
inserting a suture passer proximate to the soft tissue, the suture passer including an elongate body, a first jaw bent or bendable relative to the elongate body, and a second jaw that is distally extendable relative to a long axis of the elongate body;
positioning the first jaw of the suture passer against the soft tissue by adjusting an angle of the first jaw relative to the elongate body;
extending a sharp pointed tip of the second jaw through the soft tissue;
extending and retracting a tissue penetrator from the first jaw or the second jaw of the suture passer, and through the soft tissue between the first and second jaws to pass a suture between the first and second jaws; and
removing the suture passer from the soft tissue while leaving the suture in the soft tissue.

15. A suture passer apparatus for passing a suture, the apparatus comprising:
an elongate body extending distally and proximally along a long axis;
a first jaw extending from a distal end region of the elongate body wherein the first jaw is hinged to pivot and form an angle relative to the long axis at a hinge point with the elongate body;
a second jaw having a sharp, tissue penetrating distal tip, wherein the distal end region of the second jaw is curved toward the first jaw and includes an arcuate channel therethrough; and
a tissue penetrator configured to carry a suture, the tissue penetrator configured to extend from the arcuate channel and out of the second jaw towards the first jaw at an angle relative to the long axis.

16. The apparatus of claim 15 wherein the second jaw is configured to extend and retract proximally and distally in the long axis relative to the elongate body.

17. The apparatus of claim 15, wherein the second jaw is configured to retract completely into the elongate body.

18. The apparatus of claim 15, wherein the first jaw is configured to have a neutral position in line with the long axis of the elongate body.

19. The apparatus of claim 15, further comprising a deflection surface on the first jaw configured to deflect the tissue penetrator proximally relative to the first jaw.

20. The apparatus of claim 15, wherein the first jaw is curved.

21. The apparatus of claim 15, wherein the first jaw comprises a channel for receiving the tissue penetrator.

22. The apparatus of claim 15, wherein the first jaw comprises a suture retainer configured to retain the suture from the tissue penetrator.

23. The apparatus of claim 15, wherein the first jaw is configured to retain a tip of the tissue penetrator so that is does not extend laterally beyond the first jaw.

24. The apparatus of claim 15, wherein the tissue penetrator comprises a sharp distal tip comprising a hook region.

25. A suture passer apparatus for passing a suture, the apparatus comprising:
an elongate body extending distally and proximally along a long axis;
a curved first jaw extending from a distal end region of the elongate body, wherein the first jaw is hinged to pivot relative to the long axis of the elongate body;
a second jaw having a sharp, tissue penetrating distal tip, wherein the distal end region of the second jaw is curved toward the first jaw and includes an arcuate channel therethrough; and
a tissue penetrator housed within the second jaw and configured to carry a suture, the tissue penetrator configured to extend from the arcuate channel and out of the second jaw towards the first jaw at an angle of less than 90 degrees relative to the long axis;
a handle at the proximal end of the elongate body having a control for controlling pivoting of the curved first jaw; and
a deflection surface on the first jaw configured to deflect the tissue penetrator proximally relative to the first jaw.

26. A method of re-approximating tissue using a suture passer having an elongate body, a first jaw, and a second jaw, the method comprising:
positioning the suture passer proximate to a first tissue;
piercing the first tissue with a sharp tip of the second jaw of the suture passer;
positioning the second jaw of the suture passer adjacent a second tissue;
extending and retracting a tissue penetrator through the second tissue between the first and second jaws to pass a suture between the first and second jaws while the first tissue is pierced by the second jaw; and
pulling the second jaw out of the first tissue while leaving the suture in the first tissue and second tissue.

27. The method of claim 26, further comprising piercing the second tissue with the sharp tip of the second jaw before extending and retracting the tissue penetrator through the second tissue.

28. The method of claim 26, wherein positioning is performed minimally invasively.

29. The method of claim 26, further comprising pulling the pierced first tissue towards the second tissue with the suture passer.

30. The method of claim 26, further comprising adjusting the angle of the first jaw relative to the elongate body by pivoting the first jaw relative to the elongate body.

31. The method of claim 26, further comprising sliding the second jaw from the elongate body to extend the sharp pointed tip.

32. The method of claim 26, wherein the first tissue comprises a first region of hip capsule and the second tissue comprises a second region of hip capsule.

33. The method of claim 26, wherein pulling the second jaw out of the second tissue and the first tissue comprises withdrawing the second jaw into the elongate body to retract the sharp pointed tip.

34. The method of claim 26, wherein piercing the first tissue with the sharp tip of the second jaw comprises piercing the first tissue with a curved distal end region of the second jaw.

35. A method of minimally invasively re-approximating tissue using a suture passer having an elongate body, a first jaw, and a second jaw, the method comprising:
positioning the suture passer proximate to a first tissue;
piercing the first tissue with a sharp tip of the second jaw of the suture passer;
pulling the pierced first tissue towards a second tissue with the suture passer;
positioning the second jaw immediately adjacent to the second tissue;
adjusting the angle of the first jaw relative to the elongate body;
extending an retracting a tissue penetrator through the second tissue between the first and second jaws to pass a suture between the first and second jaws while the first tissue is pierced by the second jaw; and
pulling the second jaw out of the second tissue and the first tissue while leaving the suture in the first tissue.
